(19) United States
(12) Patent Application Publication
(10) Pub. No.: US 2017/0367787 A1
(43) Pub. Date: Dec. 28, 2017

(54) DENTAL RESTORATION TOOL

(71) Applicant: King Abdulaziz University, Jeddah (SA)

(72) Inventor: Fahad Ibrahim FATINY, Jeddah (SA)

(73) Assignee: King Abdulaziz University, Jeddah (SA)

(21) Appl. No.: 15/189,540

(22) Filed: Jun. 22, 2016

(51) Int. Cl. A61C 3/08; A61B 34/00; A61B 17/00

(52) U.S. Cl. CPC A61C 3/08; A61B 34/76; A61B 2017/00199; A61B 2017/00128; A61B 2017/0023; A61B 2017/00132

(57) ABSTRACT

A dental instrument having first portion and a second portion connected to the first end of the first portion by a connection. The connection is between a protrusion and an indentation, the first end of the first portion comprising one of a protrusion or an indentation, the first end of the second portion comprising one of a protrusion or an indentation corresponding to the first end of the first portion to allow the first portion and the second portion to connect. The connection between the first portion is rotationally adjustable relative to the second portion, each of the first portion and the second portion comprising at a second end one of a lobed portion having a lobed tool or a blade portion having a carving edge, the lobed tool having at least one lobe.

### BACKGROUND

### Field of the Disclosure

The present disclosure is directed toward a dental restoration apparatus.

### Description of the Related Art

Clinical performance of dental composites has been significantly improved over the past decade through modifications in formulation that include the use of more stable polymerization promoters for greater color stability, incorporation of high concentrations of finely ground fillers to produce adequate strength and excellent wear resistance while retaining translucency, the addition of radio pacifying agents for improved diagnostics, and utilization of dentin adhesives.

However, there are limitations to the use of composites, especially in posterior teeth. The materials remain very technique-sensitive, due to the extensive contraction which accompanies polymerization and negatively influences marginal sealing. In addition, the materials are generally considered to have inadequate mechanical properties and wear resistance in contact areas to serve as total replacements for amalgams. Efforts are focused on several areas, including the development of non- or minimally-shrinking dental composites containing spiro-orthocarbonates as additives to dimethacrylates or epoxy-base resins, the production of alternative filler materials for ideal wear resistance and aesthetics, and equipment and instruments for working with alternative filler materials.

### SUMMARY

The present disclosure is directed to a dental instrument having a first portion with a first end and a second end. A second portion has a first end connected to the first end of the first portion by a connection. The connection is between a protrusion and an indentation, the first end of the first portion having either a protrusion or an indentation. The first end of the second portion has either a protrusion or an indentation corresponding to the first end of the first portion to allow the first portion and the second portion to connect, such that the connection between the first portion is rotationally adjustable relative to the second portion. Each of the first portion and the second portion has a second end one that is either a lobed portion having a lobed tool or a blade portion having a carving edge. The lobed tool has at least one lobe.

The present disclosure is further directed to a dental instrument having a body with a first end and a second end. A first portion having a first end is connected to the first end of the body by a first connection. A second portion having a first end is connected to the second end of the body by a second connection. The first connection is between a protrusion and an indentation, the first end of the first portion having either a protrusion or an indentation, and the first end of the body having either a protrusion or an indentation corresponding to the first end of the first portion. This allows the first portion and the body to connect, such that the connection between the first portion is rotationally adjustable relative to the body. The second connection is between a protrusion and an indentation, the first end of the second portion having either a protrusion or an indentation, the second end of the body having either a protrusion or an indentation corresponding to the first end of the second portion to allow the second portion and the body to connect, such that the connection between the second portion is rotationally adjustable relative to the body. Each of the first portion and the second portion has at a second end either a lobed portion having a lobed tool or a blade portion having a carving edge. The lobed tool has at least one lobe. The body further has circuitry connected to at least one button. The circuitry has a timer activated by at least one button to track elapsed time and provide at least one signal after a specific time interval has passed.

The foregoing general description of the illustrative implementations and the following detailed description thereof are merely exemplary aspects of the teachings of this disclosure, and are not restrictive.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings wherein:

FIG. 1A is a perspective view of a dental instrument, according to one example;

FIG. 1B is a plan view of the dental instrument, according to one example;

FIG. 1C is a side view of the dental instrument, according to one example;

FIG. 2A is a side view of a blade portion and a lobed portion, according to one example;

FIG. 2B is a side view of the blade portion and the lobed portion, according to one example;

FIG. 3A is a side view of a section of the lobed portion, according to one example;

FIG. 3B is a perspective view of a lobed tool, according to one example;

FIG. 3C is a perspective view of the lobed tool in contact with a posterior tooth, according to one example;

FIG. 4A is a side view of a portion of the blade portion, according to one example;

FIG. 4B is a side view of a contoured blade, according to one example;

FIG. 4C is a perspective view of a carving edge of the contoured blade in contact with an anterior tooth, according to one example;

FIG. 5A is a plan view of a three piece dental instrument, according to one example;

FIG. 5B is a side view of the three piece dental instrument, according to one example;

FIG. 6A is a plan view of the three piece dental instrument in an unassembled state;

FIG. 6B is a plan view of the three piece dental instrument in an unassembled state;

FIG. 6C is a front view of the blade portion, according to one example;

FIG. 6D is a front view of the blade portion, according to one example;

FIG. 6E is a front view of the blade portion, according to one example;

FIG. 6F is a front view of the blade portion, according to one example;

FIG. 6G is a front view of the blade portion, according to one example;

FIG. 7A is a side view of a lobed portion of a dental instrument, according to one example;

FIG. 7B is a perspective view of a section of a lobed tool, according to one example;

FIG. 7C-FIG. 7E are each perspective views of the lobed tool in contact with a posterior tooth, according to one example;

FIG. 8A is a perspective view of the dental instrument, according to one example;

FIG. 8B is a plan view of the dental instrument, according to one example;

FIG. 8C is a side view of the dental instrument, according to one example;

FIG. 8D is a perspective view of a contoured blade in contact with an anterior tooth, according to one example; and

FIG. 8E is a perspective view of the contoured blade in contact with an anterior tooth, according to one example.

### DETAILED DESCRIPTION OF THE EMBODIMENTS

In the drawings, like reference numerals designate identical or corresponding parts throughout the several views. Further, as used herein, the words “a”, “an” and the like generally carry a meaning of “one or more”, unless stated otherwise.

Referring now to the drawings, wherein like reference numerals designate identical or corresponding parts throughout the several views.

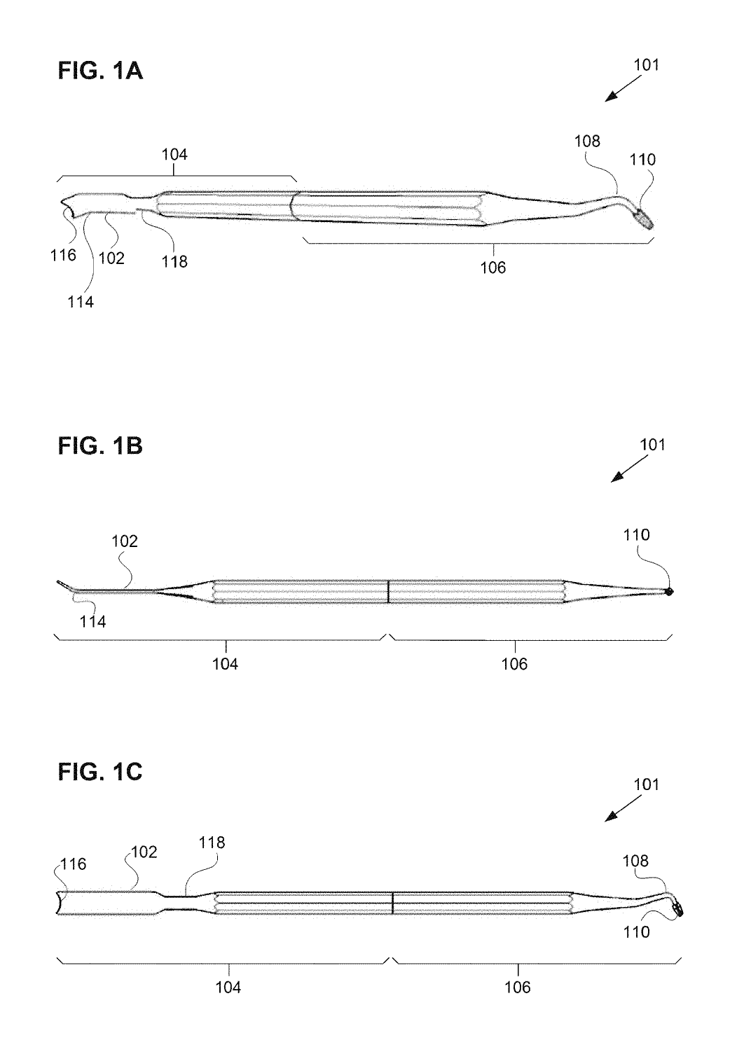

FIG. 1A is a perspective view of a dental instrument 101, according to one example. The dental instrument 101 comprises a blade portion 104 connected to a lobed portion 106. A first end of the blade portion 104 is removably connected to a first end of the lobed portion 106. A second end of the blade portion 104 comprises a contoured blade 102 having a carving edge 116 at the distal end, and a stem 118 at a proximal end, the stem 118 connecting the contoured blade 102 to the second end of the blade portion 104. A second end of the lobed portion 106 comprises a lobed tool 110, the lobed tool 110 connected to the lobed portion 106 by a curved neck 108.

The lobed tool 110 of the lobed portion 106 may be used to perform dental procedures such as manipulating uncured resin during the filling and shaping of dental cavities. Resin may be a composite. The lobed tool 110 may be formed of an adherent resistant material that functions to decrease adherence interference with uncured resin, such as during withdrawal of the dental instrument 101, and particularly the lobed tool 110, away from a tooth receiving dental care. The lobed tool 110 may displace uncured resin in a uniform and predictable way, capable of minutely displacing uncured resin through a protective sheath or emulsion. The lobed tool 110 allows for insertion of uncured resin into and adaptation of uncured resin about a tooth while reducing pullback of uncured resin upon withdrawal of the lobed tool 110 from the site of uncured resin administration. Non-impressive, low-adherence properties allow for micro-manipulation of uncured resin, leaving little or no discernable impression or “footprint” in the uncured resin, saving time and producing more desirable functional and cosmetic end results. (Cosmetic end results are of particular importance with anterior teeth.) Further, particularly for posterior teeth, an ability to more precisely manipulate uncured resin, such as through seamless and ultrathin feathering, may allow for improved accuracy of teeth alignment between teeth of an upper jaw and teeth of a lower jaw. Further, the lobed tool 110 may be coated with a coating, such as Teflon, titanium nitride, or Parylene, for less adherence in use with uncured resins. The lobed tool 110 may be formed of at least one lobe 112 that comprises a surface texture, for example, such as a texture with dimples, concave depressions, convex protrusions to aid with reduced adherence during use with non-stick composites and resins.

The contoured blade 102 of the blade portion 104 may be used for removal of excess composite or resin cement from a tooth, for example after a cavity filling procedure. A carving edge 116 of the contoured blade 102 may be used on the curved interproximal surfaces of a tooth, the carving edge 116 may be disposed at an angle with respect to the contoured blade 102 via an ergonomic bend 114. The contoured blade 102 may be flat, and the carving edge 116 may have a narrow width and a radial curvature that resembles that of a portion of an anterior tooth surface, for scraping excess resin material from a tooth. In one example, the carving edge 116 is under 12 mm in width. In another example, the carving edge 116 is in the range of 8 mm to 12 mm in width, such as to accommodate larger anterior tooth surfaces. In another example, the carving edge 116 is in the range of 3 mm to 8 mm in width, such as to accommodate smaller tooth surfaces of children. In another example, the carving edge 116 is less than 3 mm in width, such as to access less accessible surfaces disposed on posterior teeth.

Sharpness of the carving edge 116 may vary depending on the end needed, such as a sharp edge or a thicker edge for chiseling. In one example, the carving edge 116 has a thickness of under 0.25 mm at the leading edge. In another example, the carving edge 116 has a thickness of under 0.45 mm at the leading edge. In another example, the carving edge 116 has a thickness under 0.55 mm at the leading edge. In another example, the carving edge 116 is a blunt surface with a thickness in the range of between 0.55 mm and 1.00 mm at the leading edge, and may be used for removal of a fragment quantity of material on a tooth surface.

In one example, the ergonomic bend 114 comprises an angle of substantially 45 degrees with the contoured blade 102. In another example, the ergonomic bend 114 comprises an angle of 45 to 55 degrees with the contoured blade 102. In another example, the ergonomic bend 114 may comprise an angle of 46 to 51 degrees with the contoured blade 102. In another example, the ergonomic bend 114 comprises an angle of 35 to 45 degrees with the contoured blade 102. In another example, the ergonomic bend may comprise an angle of 37 to 44 degrees with the contoured blade 102.

Further, the dental instrument 101 may include two blade portions 104 disposed at opposite ends to one another where a first blade portion 104 is configured such that the ergonomic bend 114 of the first blade portion 104 is angled for ergonomic use on a first side of a tooth and a second blade portion 104 is configured such that the ergonomic bend 114 of the second blade portion 104 is angled for ergonomic use on a second side of the same tooth.

The blade portion 104 and the lobed portion 106 may each further comprise a coating 300 along at least some length of the blade portion 104 or the lobed portion 106. The coating 300 may comprise natural or artificial flavorants and be activated by fluid, such as human saliva, to provide a sensory impression of food, for example various fruits, candies, and chewing gum, or other substances, when the blade portion 104 or the lobed portion 106 is in a patient's mouth and comes into contact with human saliva.

In one example, the blade portion 104 and the lobed portion 106 may each be disposable after an one-time, single use. In another example, the blade portion 104 and the lobed portion 106 may be disposable after more than one use. In another example, the blade portion 104 and the lobed portion 106 may be reusable. In a case where the blade portion 104 and the lobed portion 106 are reusable, the coating 300 may not be as durable as the blade portion 104 and the lobed portion 106, such that after a number of uses the blade portion 104 and the lobed portion 106 may no long possess any discernable flavor from the coating 300.

FIG. 1B is a plan view of the dental instrument 101, according to one example. In this view the dental instrument 101 comprises the blade portion 104 and the lobed portion 106 as described by FIG. 1A.

FIG. 1C is a side view of the dental instrument 101, according to one example. In this view the dental instrument 101 comprises the blade portion 104 and the lobed portion 106 as described by FIG. 1A and FIG. 1B.

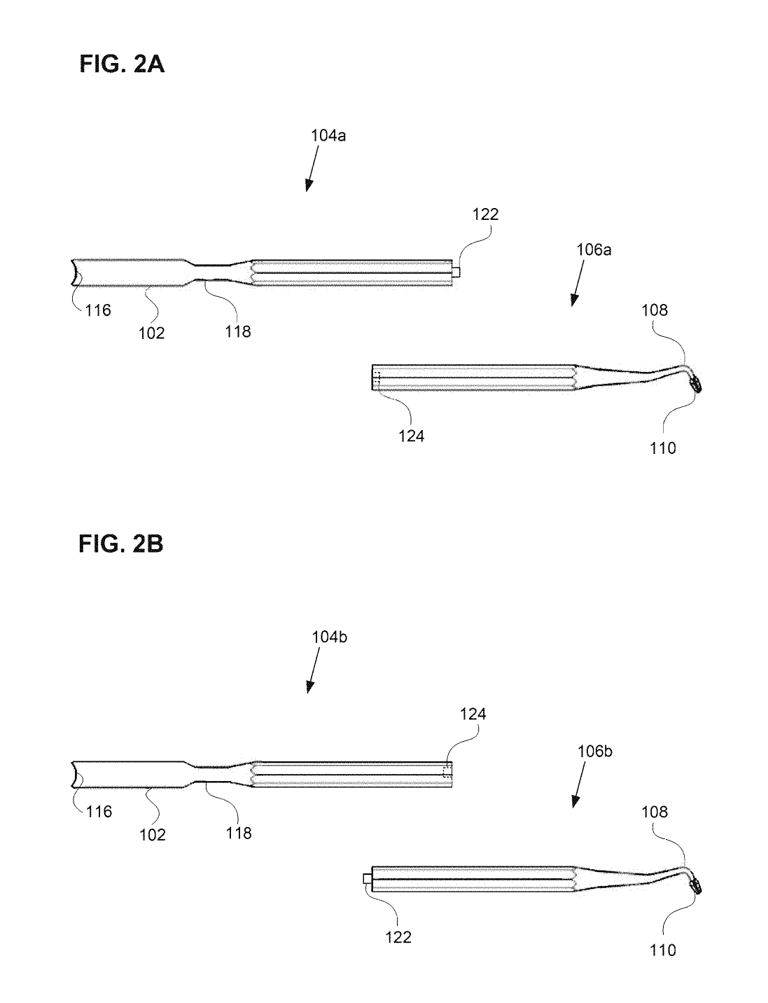

FIG. 2A is a side view of the blade portion 104a and the lobed portion 106a in an unassembled state, according to one example. The first end of the blade portion 104a and the first end of the lobed portion 106a may be separated to reveal a protrusion 122 disposed on the first end of the blade portion 104a and an indentation 124 disposed within the first end of the lobed portion 106a. The protrusion 122 has approximately the same corresponding size and profile of the indentation 124, allowing the protrusion 122 to fit within the indentation 124 and connect the blade portion 104a to the lobed portion 106a such that the blade portion 104a and the lobed portion 106a may form a single dental instrument 101 when connected.

Each protrusion 122 and corresponding indentation 124 may lock by friction or such that they securely snap together when pressed to form the dental instrument 101. The protrusion 122 and the indentation 124 may comprise a variety of corresponding shapes, for example cubic or hemispherical shapes. In another example, the protrusion 122 may comprise a threaded screw and the indentation 124 may comprise a threaded cylinder that corresponds to the threaded screw. In another example, the protrusion 122 may comprise a non-threaded, elongated rod and the indentation 124 may comprise a non-threaded cylinder that corresponds to the threaded screw, and at least one of the protrusion 122 and the indentation 124, and may further comprise a reverse taper in the region where the protrusion 122 is nearly fully inserted into the indentation 124 resulting in an interlock to secure the blade portion 104 to the lobed portion 106.

Each blade portion 104 and lobed portion 106 may be of similar length, for example in the range of 50 mm to 76 mm, resulting in a combined length of the dental instrument 101 being in the range of 100 to 152 mm. In another example, the dental instrument 101 is in the range of 140 mm to 160 mm. Further, the diameter of the blade portion 104 and the lobed portion 106 along the widest section of each may be in the range of 5 mm to 10 mm. In another example, the diameter of the blade portion 104 and the lobed portion 106 along the widest section of each is in the range of 8 mm to 12 mm.

FIG. 2B is a side view of the blade portion 104b and the lobed portion 106b in an unassembled state, according to one example. The first end of the blade portion 104b and the first end of the lobed portion 106b may be separated to reveal an indentation 124 disposed within the first end of the blade portion 104b and a protrusion 122 disposed on the first end of the lobed portion 106b. The protrusion 122 is has approximately the same corresponding size and profile of the indentation 124, allowing the protrusion 122 to fit within the indentation 124 and connect the lobed portion 106b to the blade portion 104b such that the blade portion 104b and the lobed portion 106b may form a single dental instrument 101 when connected.

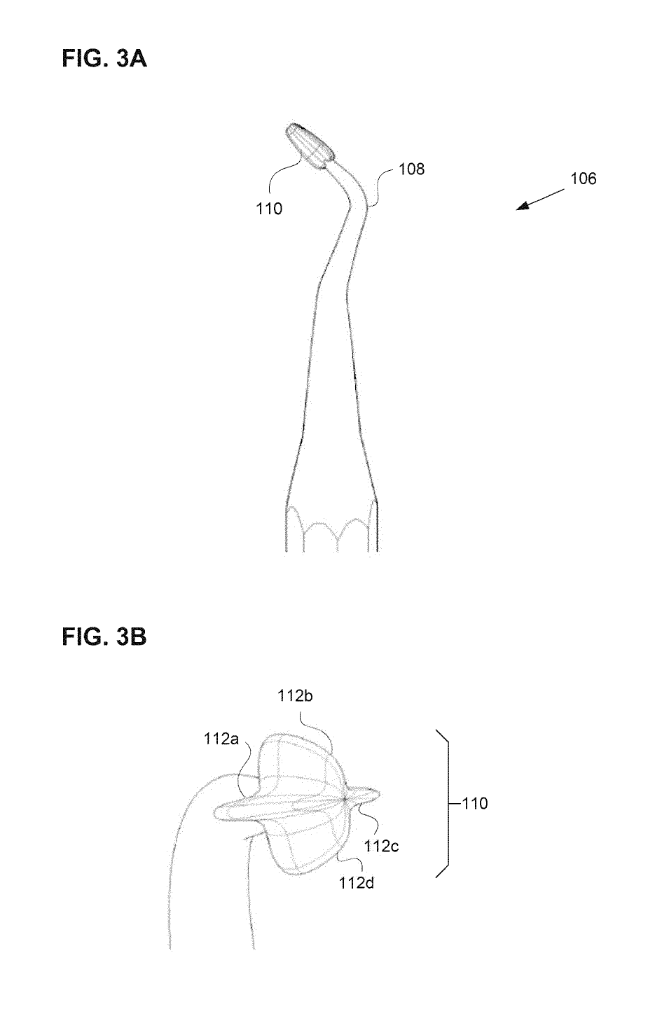

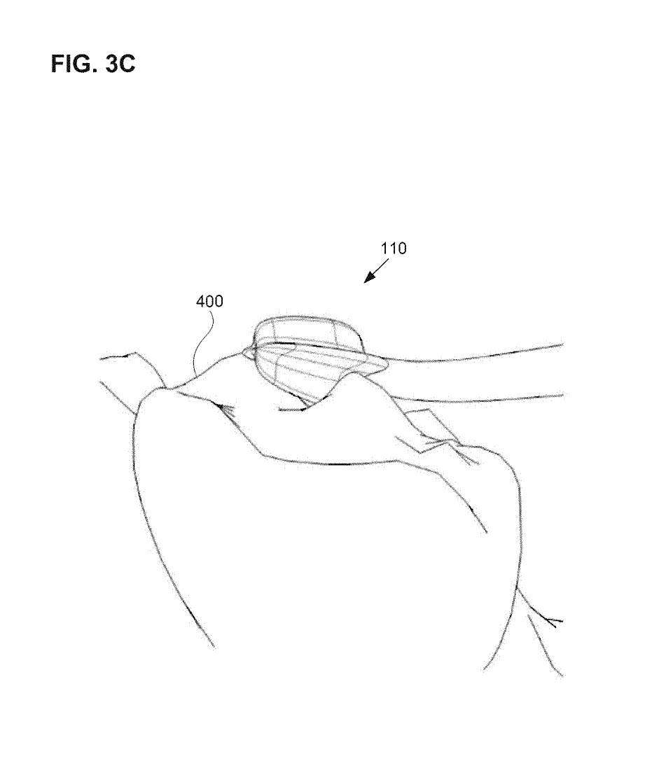

FIG. 3A is a side view of a section of the lobed portion 106, according to one example. The portion of the lobed portion 106 includes the second end of the lobed portion 106 comprising the lobed tool 110 and the curved neck 108. The lobed tool 110 is disposed at the second end of the lobed portion 106 and is connected to the lobed portion 106 by the curved neck 108. The curved neck 108 is disposed between the lobed tool 110 and the lobed portion 106.

FIG. 3B is a perspective view of the lobed tool 110, according to one example. The lobed tool 110 comprises at least one lobe 112, and the at least one lobe 112 may be compressible that that does not adhere to uncured resin. In one example, the lobed tool 110 may have a cruciform shape comprising four lobes 112 (112a-112d) with undulating serrated edges for distributing, compressing, shaping, and manipulating uncured resin. In one example, the lobed tool 110 comprises four lobes 112. In another example, the lobed tool 110 may comprise three lobes 112. In another example, the lobed tool 110 may comprise five lobes 112. The lobed tool 110 is intended to manipulate yet minimize adhesion to uncured resin to fill teeth cavities, and at least a portion of the lobed tool 110 may be formed of a variety of materials such as compressible foam, polymer, or rubber compounds. Further, the lobed tool 110 may be coated or encased, for example with a non-stick coating such as Teflon, titanium nitride, or Parylene to reduce stickiness to uncured resin.

FIG. 3C is a perspective view of the lobed tool 110 in contact with a posterior tooth 400, according to one example. The lobes 112 of the lobed tool 110 are designed to allow a user to navigate the undulations of surfaces of the posterior tooth 400 to perform dental procedures.

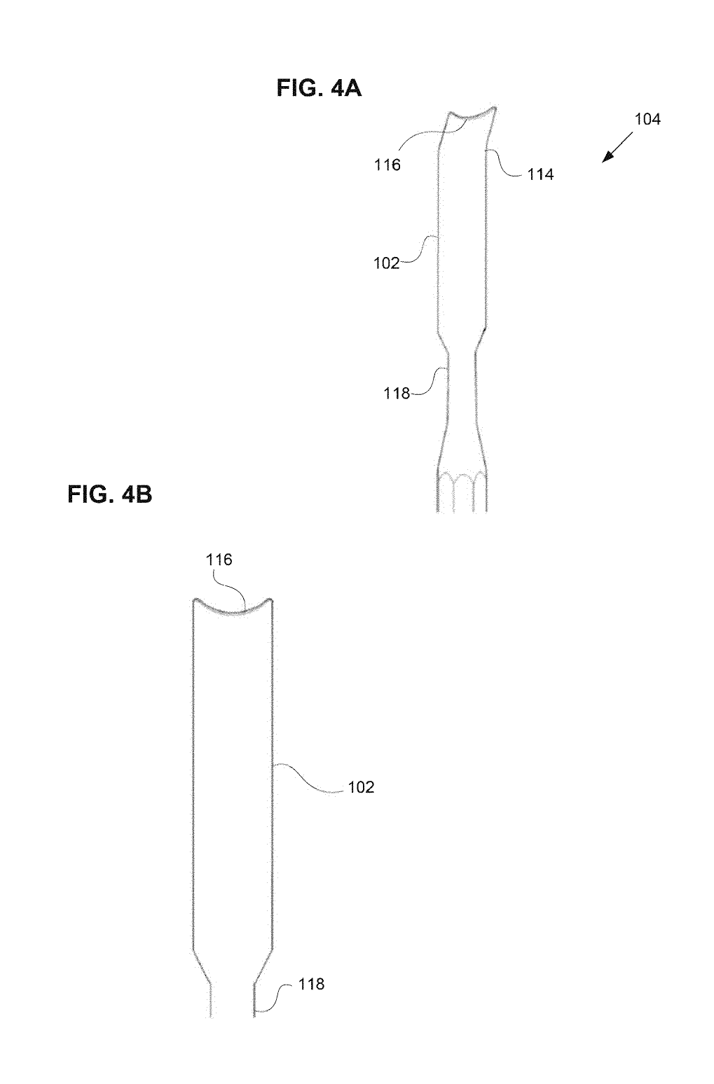

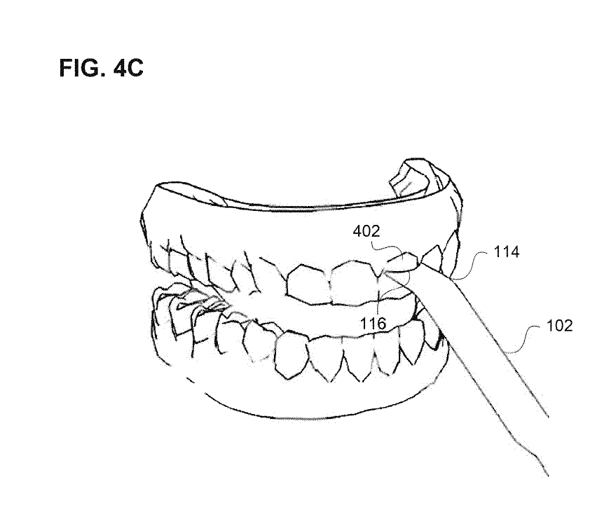

FIG. 4A is a side view of a portion of the blade portion 104, according to one example. The portion of the blade portion 104 includes the second end of the blade portion 104 comprising the contoured blade 102. The contoured blade 102 is disposed at the second end of the blade portion 104 and is connected to the blade portion 104 by the stem 118. The stem 118 is disposed between the contoured blade 102 and the first end of the blade portion 104, and may be quite thin, for example in the range of 3 mm to 7 mm to maximize maneuverability while providing adequate strength, and have a substantially circular or elliptical cross section. In other examples may comprise a square, pentagonal, or hexagonal cross section. The contoured blade 102 further comprises the ergonomic bend 114 and the carving edge 116.

The blade portion 104 and the lobed portion 106 may be sterilized in an autoclave and may be formed of disposable plastic or reusable metal, such as stainless steel, carbon steel, and the blade portion 104 and the contoured blade 102 may further comprise materials such as tungsten carbide.

FIG. 4B is a side view of the contoured blade 102, according to one example. A first end of the contoured blade 102 is connected to the stem 118 and a second end of the contoured blade 102 comprises the carving edge 116. The contoured blade 102 may be a flat plane, with the carving edge 116 disposed at the second end and having a curvature in only one plane. The curvature of the carving edge 116 may have a radius or compound radius approximating that of an anterior tooth 402, for example in the range of 10 mm to 12 mm. In another example, the radius is in the range of 12 mm to 18 mm. In another example, the radius is in the range of 18 mm to 26 mm. The range of radii of the carving edge 116 allow a user to select the best suited curvature, depending on tooth size, surface, and location within a dental arch.

FIG. 4C is a perspective view of the carving edge 116 of the contoured blade 102 in contact with an anterior tooth 402, according to one example. The curvature of the carving edge 116 is designed to approximate a curvature of the anterior tooth 402, allowing an user to scrape the anterior tooth 402 to perform a dental procedure, such as removal of excess resin. Further, the contoured blade 102 comprises the ergonomic bend 114 that may allow more ergonomic access to the anterior tooth 402.

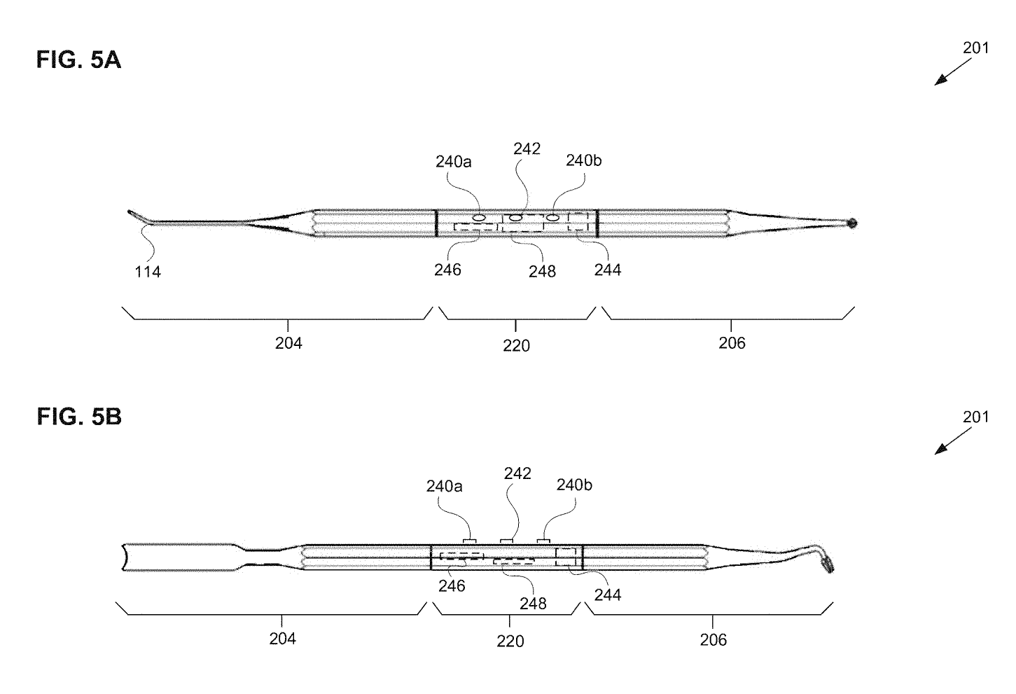

FIG. 5A is a plan view of a three piece dental instrument 201, according to one example. The three piece dental instrument 201 comprises an instrument body 220 connected at a first end to a blade portion 204 and connected at a second end to a lobed portion 206. The instrument body 220 may further comprise a circuitry 244 connected to an energy storage device 246 that provides energy for the instrument body 220 to operate. The circuitry 244 may be further connected to at least one button 240 and/or connected to at least one indicator light 242, and a display 248, such as for indicating to a user a resin cure time during a dental procedure.

In one example, pressing the button 240a may begin a countdown for a first preset amount of time.

In another example, pressing the button 240b may begin a countdown for a second preset amount of time.

In another example, pressing both the button 240a and the button 240b simultaneously allows an user to adjust a duration of the first or the second time interval, with a change in the first or the second interval during adjustment may be indicated by an audible or a haptic signal or pattern of signals emitted by the circuitry 244 or a visual light signal emitted by the indicator light 242. For example, audible signals may include singular or a series of chirps, beeps, and buzzing, while haptic signals may include vibration of the instrument body 220, once or multiple times at various intervals.

In another example, light signals may include the indicator light 242 remaining illuminated from the time a button 240a or a button 240b is depressed until the first or the second preset time interval has passed.

In another example, light signals may include the indicator light 242 becoming illuminated once the first or the second preset time interval has passed from the initial depression of the button 240a or the button 240b.

In another example, light signals may include the indicator light 242 illuminating multiple times once the first or the second preset time interval has passed from the initial depression of the button 240a or the button 240b.

In one example, the blade portion 204 is identical to the blade portion 104a, with the exception that the length of the body of the blade portion 204 may be shorter than that of the blade portion 104a. The lobed portion 206a is identical to the lobed portion 106a, with the exception that the length of the body of the lobed portion 206 may be shorter than that of the lobed portion 106a.

In another example, the blade portion 204 is identical to the blade portion 104b, with the exception that the length of the blade portion 204 may be shorter than that of the blade portion 104b, and the lobed portion 206 is identical to the lobed portion 106b, with the exception that the length of the lobed portion 206 may be shorter than that of the lobed portion 106b.

In another example, the instrument body 220 may further comprise a display 248. The display 248 allows information to be displayed, for example a time or a time interval. An increase or decrease in the time or the time interval may be displayed. Simultaneous depression of both the button 240a and the button 240b may allow adjustment of the time interval, for example depression of the button 240a may increase the time interval while depression of the button 240b may decrease the time interval.

FIG. 5B is a side view of the three piece dental instrument 201, according to one example. In this view the three piece dental instrument 201 comprises an instrument body 220 connected at a first end to a blade portion 204 and connected at a second end to a lobed portion 206, as described by FIG. 5A.

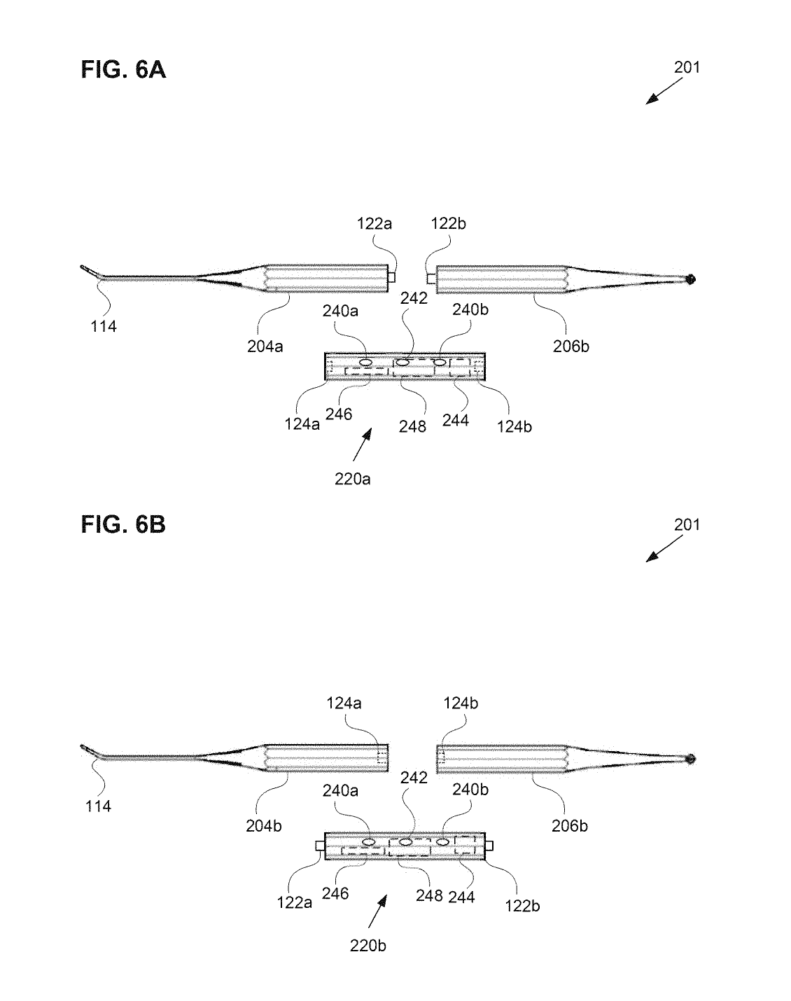

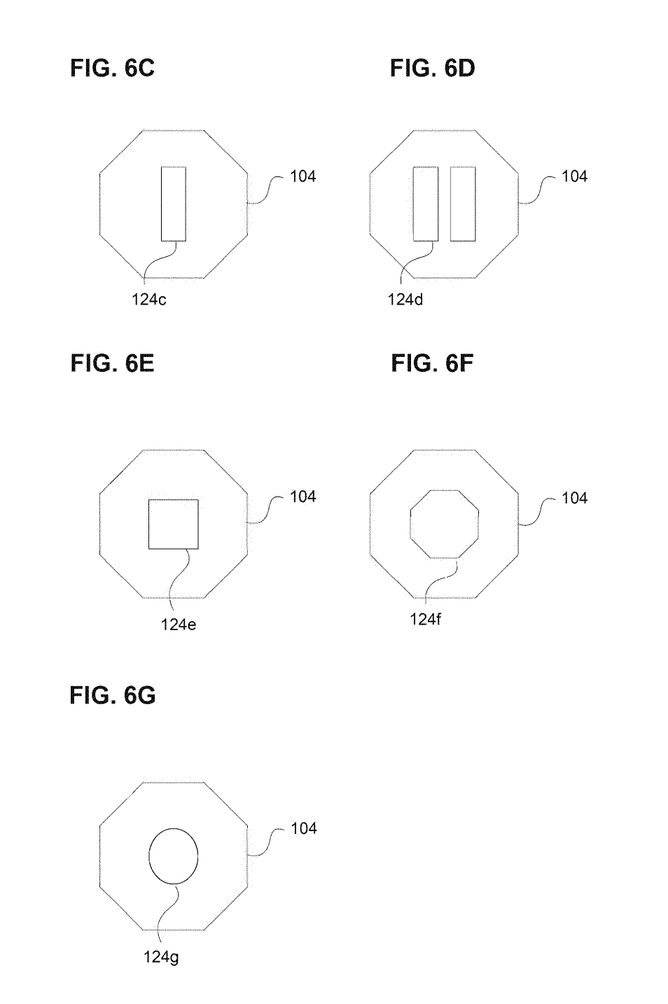

FIG. 6A is a plan view of the three piece dental instrument 201 in an unassembled state, according to one example. The body 220a further comprises an indentation 124a disposed within the first end and an indentation 124b disposed within the second end. The indentation 124a and the indentation 124b may be identical.

The blade portion 204a further comprises a protrusion 122a disposed on a first end of the blade portion 204a, and the lobed portion 206a further comprises a protrusion 122b disposed on a first end of the lobed portion 206a. The protrusion 122a and the protrusion 122b may be identical. The protrusion 122a has approximately the same corresponding size and profile of the indentation 124a, allowing the protrusion 122a to fit within the indentation 124a and connect the first end of the blade portion 204a to a first end of the body 220a. The protrusion 122b has approximately the same corresponding size and profile of the indentation 124b, allowing the protrusion 122b to fit within the indentation 124b and connect the first end of the lobed portion 206a to a second end of the body 220a.

FIG. 6B is a plan view of the three piece dental instrument 201 in an unassembled state, according to one example. The body 220b further comprises the protrusion 122a disposed on the first end and the protrusion 122b disposed on the second end, while the blade portion 204b further comprises an indentation 124a disposed within a first end of the blade portion 204b, and the lobed portion 206b further comprises an indentation 124b disposed within a first end of the lobed portion 206b. The protrusion 122a and the protrusion 122b may be identical. The protrusion 122a has approximately the same corresponding size and profile of the indentation 124a, allowing the protrusion 122a to fit within the indentation 124a and connect the first end of the blade portion 204b to a first end of the body 220b. The protrusion 122b has approximately the same corresponding size and profile of the indentation 124b, allowing the protrusion 122b to fit within the indentation 124b and connect the first end of the lobed portion 206b to a second end of the body 220b.

FIG. 6C-FIG. 6G represent connections between the protrusion 122 and the indentation 124. For example, the blade portion 104a comprises the protrusion 122 at the first end to connect the blade portion 104a to the first end of the lobed portion 106a, the lobed portion 106a comprising the indentation 124 disposed within the first end of the lobed portion 106a.

In another example, the protrusion 122 and the indentation 124 connect the blade portion 104b, the blade portion 104b comprising the indentation 124 disposed within the first end of the blade portion 104b, and the first end of the lobed portion 106b, the lobed portion 106b comprising the protrusion 122 disposed at the first end of the lobed portion 106b.

In another example, the protrusion 122a and the indentation 124a connect the blade portion 204a, the blade portion 204a comprising the protrusion 122a disposed on the first end of the blade portion 204a, and the body 220a, the indentation 124a disposed within the first end of the body 220a. The protrusion 122b and the indentation 124b connect the lobed portion 206a, the lobed portion 206a comprising the protrusion 122b disposed on the first end of the lobed portion 206a, and the body 220a, the body 220a comprising the indentation 124b disposed within the second end of the body 220a.

Further, any protrusion 122 may be connectable to a corresponding indentation 124, regardless of what portion described the protrusion 122 and the indentation 124 are disposed on or within. In one example, in this way a first instrument body 220 may be connectable to at least one of a blade portion 104, a lobed portion 106, a blade portion 204, a lobed portion 206, and a second instrument body 220.

In another example, a first blade portion 104 may be connectable to at least one of a second blade portion 104, a lobed portion 106, a blade portion 204, and a lobed portion 206.

In another example, a first blade portion 204 may be connectable to at least one of a second blade portion 204, a lobed portion 106, a blade portion 104, and a lobed portion 206.

The same type of connections between the protrusion 122 and the indentation 124 may also be used to connect a curved neck 108 to a lobed portion 106, a blade portion 204 to an instrument body 220, or a lobed portion 206 to an instrument body 220. While the examples of FIG. 6C-FIG. 6G state the use of the blade portion 104, one having ordinary skill in the art will recognize the same may also apply to any of the lobed portion 106, the blade portion 204, the lobed portion 206, the instrument body 220. Rotational adjustment may be accomplished by the separation of two portions, such as the blade portion 104 and the lobed portion 106, rotating the blade portion 104 about its longitudinal axis relative to the lobed portion 106, and reconnecting the blade portion 104 and the lobed portion 106. Further, the amounts of rotational adjustment are not limited to the examples of FIG. 6C-FIG. 6G.

FIG. 6C is a front view of the blade portion 104c, according to one example. The blade portion 104c comprises a protrusion 122c to connect to an indentation 124c with corresponding size and profile. The connection between the protrusion 122c and the indentation 124c may allow adjustment in increments of 180 degrees between the protrusion 122c and the corresponding indentation 124c.

FIG. 6D is a front view of the blade portion 104d, according to one example. The blade portion 104d comprises a protrusion 122d to connect to an indentation 124d with corresponding size and profile. The connection between the protrusion 122d and the indentation 124d may allow adjustment in increments of 180 degrees between the protrusion 122d and the corresponding indentation 124d.

FIG. 6E is a front view of the blade portion 104e, according to one example. The blade portion 104e comprises a protrusion 122e to connect to an indentation 124e with corresponding size and profile. The connection between the protrusion 122e and the indentation 124e may allow adjustment in increments of 90 degrees between the protrusion 122e and the corresponding indentation 124e.

FIG. 6F is a front view of the blade portion 104f, according to one example. The blade portion 104f comprises a protrusion 122f to connect to an indentation 124f with corresponding size and profile. The connection between the protrusion 122f and the indentation 124f may allow adjustment in increments of 45 degrees between the protrusion 122f and the corresponding indentation 124f.

FIG. 6G is a front view of the blade portion 104g, according to one example. The blade portion 104g comprises a protrusion 122g to connect to an indentation 124g with corresponding size and profile. The connection between the protrusion 122g and the indentation 124g may allow adjustment in any rotational increment between the protrusion 122g and the indentation 124g if the shape of the protrusion 122g and the corresponding indentation 124g are circular.

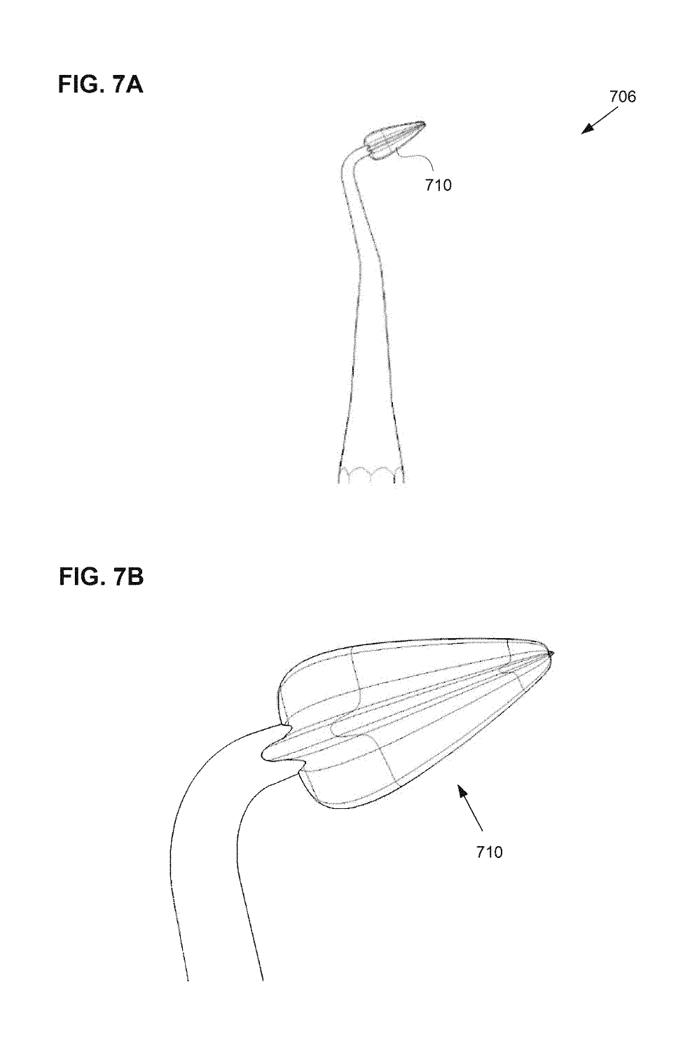

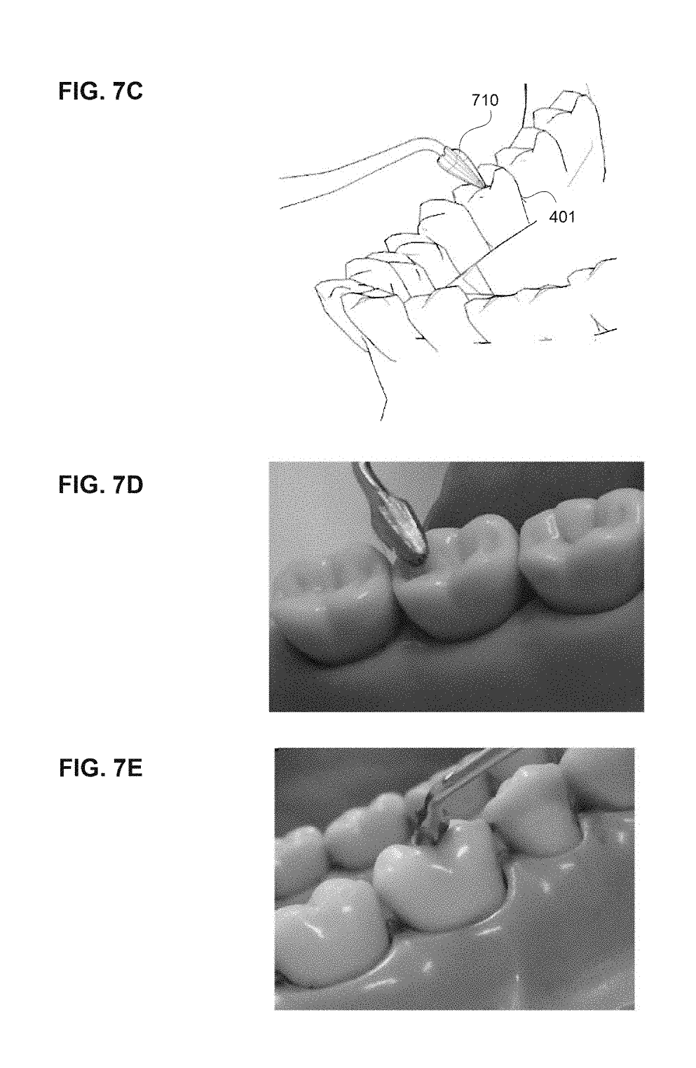

FIG. 7A is a side view of a lobed portion 706 of a dental instrument 701, according to one example. A lobed tool 710 of the lobed portion 706 may be used to perform dental procedures such as manipulating uncured resin during the filling and shaping of dental cavities, as described by that for the dental instrument 101. However, the shape of the lobed tool 710 is sharper than that of lobed tool 110 for use in applications requiring, for example, greater precision, for filling smaller cavities, or performing procedures on certain buccal, lingual, and marginal grooves.

FIG. 7B is a perspective view of a section of the lobed tool 710, according to one example. With the exception of its shape, the lobed tool 710 is identical in its construction and purpose to that of lobed tool 110 described by FIG. 1A.

FIG. 7C-FIG. 7E are each perspective views of the lobed tool 710 in contact with a posterior tooth 401, according to one example. The lobed tool 710 is designed to allow a user to navigate the undulations of surfaces of the posterior tooth 401 to perform dental procedures.

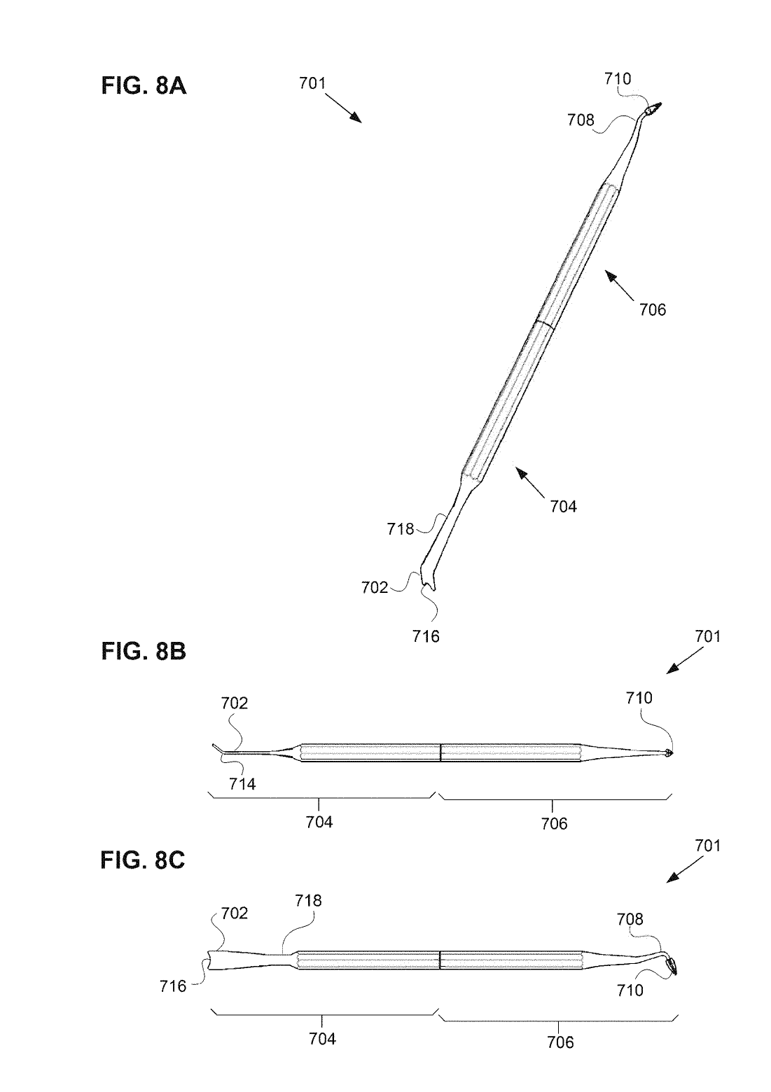

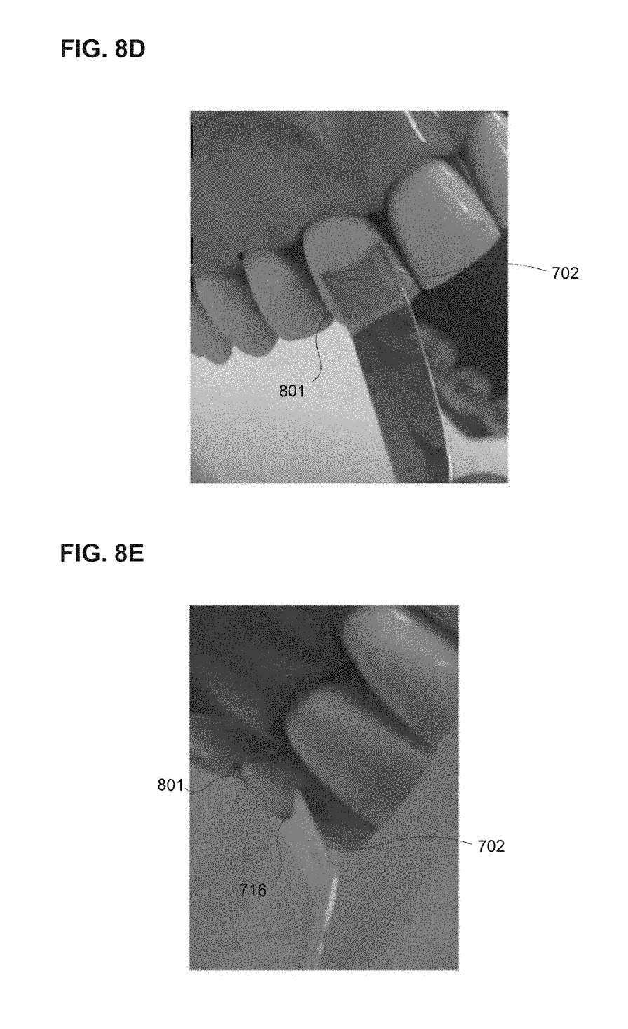

FIG. 8A is a perspective view of the dental instrument 701, according to one example. The dental instrument 701 comprises a blade portion 704 connected to the lobed portion 706. A first end of the blade portion 704 may be removably connected to a first end of the lobed portion 706. A second end of the blade portion 704 comprises a contoured blade 702 having a carving edge 716 at the distal end, and a stem 718 at a proximal end, the stem 718 connecting the contoured blade 702 to the second end of the blade portion 704. A second end of the lobed portion 706 comprises the lobed tool 710, the lobed tool 710 connected to the lobed portion 706 by a curved neck 708. With the exception of the differences in shape between the lobed tool 110 and the lobed tool 710, as described by FIG. 7A, the dental instrument 701 is identical to the dental instrument 101.

FIG. 8B is a plan view of the dental instrument 701, according to one example. In this view the dental instrument 701 comprises the blade portion 704 and the lobed portion 706, the blade portion 704 comprising a contoured blade 702 and an ergonomic bend 714, and the lobed portion 706 comprising the lobed tool 710.

FIG. 8C is a side view of the dental instrument 701, according to one example. In this view the dental instrument 701 comprises the blade portion 704 and the lobed portion 706 as described by FIG. 8A and FIG. 8B.

FIG. 8D is a perspective view of the contoured blade 702 in contact with an anterior tooth 801, according to one example. In this view the contoured blade 702 is in contact at an angle with a labial surface of the anterior tooth 801.

FIG. 8E is a perspective view of the contoured blade 702 in contact with an anterior tooth 801, according to one example. In this view the contoured blade 702 is in contact with a labial surface of the anterior tooth 801 though at a different angle than from that described by FIG. 8D, resulting in greater contact between a curvature of the carving edge 716 and the labial surface of the anterior tooth 801. By varying an angle of contact between the contoured blade 702 and the anterior tooth 801, more or less of the curvature of the carving edge 716 may be used to adapt to varying tooth morphology.

Thus, the foregoing discussion discloses and describes merely exemplary embodiments of the present application. As will be understood by those skilled in the art, the present application may be embodied in other specific forms without departing from the spirit or essential characteristics thereof. Accordingly, the disclosure of the present application is intended to be illustrative, but not limiting of the scope of the application, as well as other claims. The disclosure, including any readily discernable variants of the teachings herein, define, in part, the scope of the foregoing claim terminology such that no inventive subject matter is dedicated to the public.

1. A dental instrument comprising:
a first portion having a first end and a second end;
a second portion having a first end connected to the first end of the first portion by a connection and a second end; wherein,
the connection is between a protrusion and an indentation, the first end of the first portion comprising one of the protrusion or the indentation, the first end of the second portion comprising one of the protrusion or the indentation corresponding to the first end of the first portion to allow the first portion and the second portion to connect such that the connection between the first portion is rotationally adjustable relative to the second portion, the first portion including at the second end a lobed portion having a lobed tool, the second portion including at the second end a blade portion having a carving edge, the lobed tool having four lobes.

2. The dental instrument of claim 1 wherein:
the protrusion and the indentation have a profile configured to allow rotation in fixed angular increments.

3. The dental instrument of claim 1 wherein:
the rotationally adjustable connection between the first portion and the second portion is rotatable in any angular increment.

4. The dental instrument of claim 1 further comprising:
a flavored coating along a length of at least one of the first portion and the second portion.

5. (canceled)

6. The dental instrument of claim 1 wherein:
at least one of the first portion and the second portion is a one-time, single use portion.

7. The dental instrument of claim 1 wherein:
the first portion and the second portion include an ergonomic bend.

8. A dental instrument comprising:
a body having a first end and a second end;
a first portion having a first end connected to the first end of the body by a first connection;
a second portion having a first end connected to the second end of the body by a second connection; wherein,
the first connection is between a protrusion and an indentation, the first end of the first portion comprising one of a protrusion or an indentation, the first end of the body comprising one of a protrusion or an indentation corresponding to the first end of the first portion to allow the first portion and the body to connect, such that the connection between the first portion is rotationally adjustable relative to the body,
the second connection is between a protrusion and an indentation, the first end of the second portion comprising one of a protrusion or an indentation, the second end of the body comprising one of a protrusion or an indentation corresponding to the first end of the second portion to allow the second portion and the body to connect, such that the connection between the second portion is rotationally adjustable relative to the body,
each of the first portion and the second portion comprising at a second end one of a lobed portion having a lobed tool and a blade portion having a carving edge, the lobed tool having at least one lobe,
the body further comprising a circuitry connected to at least one button, the circuitry having a timer activated by the at least one button to track elapsed time and provide at least one signal after a specific time interval has passed.

9. The dental instrument of claim 8 further comprising:
a flavored coating along a length of at least one of the first portion, the second portion, and the body.

10. The dental instrument of claim 8 wherein:
at least one of the rotationally adjustable connections between the first portion and the body and between the second portion and the body is rotatable in fixed angular increments.

11. The dental instrument of claim 8 wherein:
at least one of the rotationally adjustable connections between the first portion and the body and between the second portion and the body is rotatable in any angular increment.

12. The dental instrument of claim 8 wherein:
the lobed tool comprises four lobes.

13. The dental instrument of claim 8 wherein:
the at least one signal is an audible sound.

14. The dental instrument of claim 8 further comprising:
at least one indicator light, wherein the at least one signal is a visual light signal.

15. The dental instrument of claim 8 wherein:
the at least one signal is a haptic signal.

16. The dental instrument of claim 8 further comprising:
a display screen; wherein, the at least one signal is an indication on the display screen.

17. The dental instrument of claim 8 wherein:
at least one of the first portion and the second portion is a one-time, single use portion.

18. The dental instrument of claim 8 wherein:
the first portion and the second portion each comprise a blade portion, the blade portion having an ergonomic bend, and the first portion is angled for ergonomic use on a first side of a tooth and the second portion is configured such that the ergonomic bend of the second blade portion is angled for ergonomic use on a second side of the same tooth.

19. The dental instrument of claim 7, wherein the ergonomic bend comprises an angle of 45 to 55 degrees.

20. The dental instrument of claim 2, wherein the profile has a hexagonal shape to allow rotation in 45 degrees increments.
